(19) United States
(12) Patent Application Publication
(10) Pub. No.: US 2026/0272868 A1
(43) Pub. Date: Sep. 17, 2026

(54) METHODS OF USING TRAZODONE TO REVERSE THE EFFECTS OF 5-HT2A RECEPTOR AGONISTS

(71) Applicant: Gilgamesh Pharmaceuticals, Inc., New York, NY (US)

(72) Inventor: Andrew Carry Kruegel, Millington, NJ (US)

(73) Assignee: Gilgamesh Pharmaceuticals, Inc., New York, NY (US)

(21) Appl. No.: 19/128,830

(22) PCT Filed: Nov. 10, 2023
(86) PCT No.: PCT/US2023/037122

§ 371 Date: May 9, 2025

Related U.S. Application Data
(60) Provisional application No. 63/383,380, filed on Nov. 11, 2022.

(51) Int. Cl. A61K 31/198; A61K 31/496

(52) U.S. Cl. CPC A61K 31/198; A61K 31/496

(57) ABSTRACT

The present disclosure relates to the administration of trazodone or a pharmaceutically acceptable salt thereof to reverse the pharmacological effects of a 5-HT2A receptor agonist in a subject.

### CROSS REFERENCE TO RELATED APPLICATION

This application claims the benefit of U.S. Provisional Patent Application No. 63/383,380, filed Nov. 11, 2022, the contents of which are incorporated herein by reference in its entirety.

### FIELD OF THE DISCLOSURE

The present disclosure relates to the use of trazodone or pharmaceutically acceptable salts thereof to reverse the effects, including hallucinogenic effects, of 5-HT2A receptor agonists.

### BACKGROUND

Psychiatric illnesses, including depression and anxiety, represent a serious detriment to health and effective human functioning worldwide. Although a number of psychiatric medications are available and extensively prescribed, they fail to deliver relief for many individuals. For those patients who do respond, changes in mood and behavior occur, but they are often slow to manifest. In recent years, these persistent unmet needs for improved pharmacotherapies to treat psychiatric disorders have led to consideration of previously maligned options. Agonists of the 5-HT2A receptor, including prototypical agents such as psilocybin, N, N-dimethyltryptamine (DMT), and lysergic acid diethylamide (LSD), induce profound perceptual distortions (aka, hallucinations, or psychedelic effects) in humans. Recently, such agents have attracted considerable interest as therapeutics for psychiatric disorders, with substantial proof-of-concept demonstrated in treating depression and related mood disorders, particularly in patients unresponsive to standard-of-care treatments. However, given the profound hallucinogenic effects of these compounds, their clinical use carries substantial risks, as some patients may experience anxiety or panic due to the intensity of the psychoactive effect. In some cases, this may lead patients to desire to abort the experience before its natural conclusion, particularly for 5-HT2A receptor agonists that have multi-hour durations of action (e.g., psilocybin). Additionally, 5-HT2A receptor agonists have effects on the cardiovascular system, and induce vasoconstriction, increases in blood pressure, and in rare cases, cardiac vasospasm. In rare cases, such effects have resulted in amputations from a loss of peripheral circulatory function and even death. Furthermore, the long duration of many 5-HT2A receptor agonists makes them inconvenient to use in supervised therapy sessions due to the substantial time commitment needed from both the patient and healthcare provider (e.g., more than four hours in the case of psilocybin).

Accordingly, a means of rapidly reversing the effects of 5-HT2A receptor agonists would be useful for both aborting negative or dangerous treatment sessions or for shortening a treatment session for convenience. The need for such a reversal agent is only expected to grow as 5-HT2A receptor agonists gain greater adoption as psychiatric treatments in the coming years.

### SUMMARY OF THE DISCLOSURE

The present disclosure has found a means for addressing this need through the administration of trazodone. Trazodone is marketed as an antidepressant, anxiolytic, and sleep aid. Typically, oral doses of ≥150 mg/day are used to treat depression, while doses as low as 50 mg/day are used to aid in sleep. Trazodone has not been used by parenteral routes beyond exploratory studies (e.g., intravenous PK studies). The drug has a complex pharmacology and binds to a number of serotonin and adrenergic receptors, exhibiting mostly antagonist actions at these targets. Among the targets of trazodone is the 5-HT2A receptor, where the compound binds with nanomolar affinity and acts as an antagonist in vitro.

Accordingly, the present disclosure provides methods of using trazodone to reverse the effects, such as the hallucinogenic effects, of 5-HT2A receptor agonists. More specifically, in an embodiment, the present disclosure relates to a method of reversing or abating the pharmacological effect from a 5-HT2A receptor agonist after cessation of the administration of said 5-HT2A receptor agonist to a subject comprising administering to the subject in need of such treatment a therapeutically effective amount of trazodone or pharmaceutically acceptable salt thereof.

### BRIEF DESCRIPTION OF THE DRAWINGS

The objects, features and advantages will become apparent to one of ordinary skill in the art, in view of the following detailed description taken in combination with the attached drawings.

FIG. 1 graphically depicts a timeline of a trazodone reversal experiment described in Example 1.

FIG. 2 graphically depicts mean HTR/min recorded from 8 mice per treatment condition, shown as 2-minute time bins in the 30-minute recording period following administration of various doses of trazodone in mg/kg, SC, to the mice and 45-75 minutes after administration of vehicle or DOPR 3.2 mg/kg, SC, to the mice, as described in Example 1. Error bars represent standard error of the mean. In (a), 0 mg/kg trazodone and 0 mg/kg DOPR were administered to the mice; in (b), 0 mg/kg trazodone and 3.2 mg/kg DOPR were administered to the mice; in (c), 1.0 mg/kg trazodone and 3.2 mg/kg DOPR were administered to the mice; in (d), 1.8 mg/kg trazodone and 3.2 mg/kg DOPR were administered to the mice; in (e), 3.2 mg/kg trazodone and 3.2 mg/kg DOPR were administered to the mice; in (f), 10.0 mg/kg trazodone and 3.2 mg/kg DOPR were administered to the mice.

FIG. 3 graphically depicts mean HTR/10 min recorded from 8 mice per treatment condition shown as 10-minute time bins in the 30-minute recording period following administration of various doses of trazodone in mg/kg, SC, as indicated, to the mice and 45-75 minutes after administration of vehicle or DOPR 3.2 mg/kg, SC, to the mice, as described in Example 1. Error bars represent standard error of the mean.

FIG. 4 graphically depicts the mean specific 5-HT2A receptor binding recorded from 5 mice per treatment condition, shown as counts per minute (cpm) per mm², measured 60 min after dosing vehicle or DOPR (0.32, 1, 3.2, or 32 mg/kg, SC) to the mice, as described in Example 2. Error bars represent standard error of the mean.

### DETAILED DESCRIPTION

The features and other details of the disclosure will now be more particularly described. Before further description of the present disclosure, certain terms employed in the specification, examples, and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

“Treating” includes any effect, e.g., lessening, reducing, modulating, or eliminating, that results in the improvement of the condition, disease, disorder, and the like.

The term “pharmaceutically acceptable carrier” or “pharmaceutically acceptable excipient” as used herein refers to any and all non-toxic solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising trazodone or a pharmaceutically acceptable salt thereof formulated together with one or more pharmaceutically acceptable carriers.

“Individual,” “patient,” or “subject” are used interchangeably and include any animal, including mammals, preferably mice, rats, other rodents, rabbits, dogs, cats, swine, cattle, sheep, horses, or primates, and most preferably humans. Trazodone and/or pharmaceutically acceptable salts thereof may be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods of the disclosure is desirably a mammal in which treatment of psychiatric disease or disorder is desired.

“Modulation” includes antagonism (e.g., inhibition), agonism, partial antagonism and/or partial agonism.

In the present specification, the term “therapeutically effective amount” means the amount of trazodone or pharmaceutically acceptable salt thereof that will elicit the reversal of the pharmacological effect of a 5-HT2A receptor agonist in a tissue, system, or animal, (e.g., mammal or human) that is being sought by the subject, researcher, veterinarian, medical doctor, or other clinician.

As used herein, the term “cessation of 5-HT2A receptor agonists” is the termination of the administration of 5-HT2A receptor agonists, as defined hereinbelow. The termination may occur after the administration of one dose of the 5-HT2A receptor agonists or more than one dose of the 5-HT2A receptor agonists prior to the administration of trazodone or pharmaceutically salt thereof, and no additional 5-HT2A receptor agonist is administered to the patient. In other words, no additional 5-HT2A receptor agonists are being administered to the patient, i.e., administration of the 5-HT2A receptor agonists is stopped.

The term “abnormal thought”, as used herein, refers to thoughts distinct from those typically occurring during normal consciousness. Examples of abnormal thoughts include, but are not limited to, confusion, thought loops, thoughts of repetitive doubts about relationships, various decisions, sexual orientation or identity, intrusions of thoughts about safety, religion, death, and weird thoughts that make no apparent sense.

As used herein, the term “visual disturbance” refers to hallucinatory visual symptoms or perception that interfere with sight resulting from the administration of a 5-HT2A receptor agonist. Examples include, but are not limited to, changes in color perception, complex patterns overlayed over the visual field, alterations in depth perception or sense of scale with objects appearing larger/smaller or closer/further than reality, trailing after lights and other objects, apparent bending breathing, or warping of objects, mirages, blurred vision, double vision, partial or total blindness, halos, i.e., circles of limit around objects or any other symptom that interferes with the ability of a subject to see.

The term “auditory disturbance”, as used herein refers to an auditory distortion or hallucination that results from the administration of a 5-HT2A receptor agonist wherein sounds perceived by the subject are distorted compared to normal perception or in which the subject hears imaginary noises that do not exist in reality at the time that the subject hears the noise but exist in the mind of the subject. For example, the subject hears whirring noises, mechanical whistling, or music when no such noise exists in reality at the time that the subject alleges that he or she hears the noise of which he or she is complaining. Alternatively, perception of real sounds may be distorted, including changes in pitch, intensity, or timbre.

As used herein, the term “perceptual disturbance” refers to alterations or distortions of normal sensory phenomena or entirely imaginary sensory phenomena that the subject perceives, but which are not based on reality.

As used herein, the term “psychedelic effects” refers to, collectively, the acute perceptual disturbances and changes in mood and behavior induced by 5-HT2A receptor agonists acting on the central nervous system during the period that they remain present in the bloodstream at pharmacologically relevant concentrations. The term “pharmacologically relevant concentrations” as used herein refer to the concentration or amount of 5-HT2A-agonist present in the bloodstream that are effective in causing or maintaining psychedelic effects in the subject.

The term “pharmaceutically acceptable salt(s)” as used herein refers to non-toxic salts of the amine basic groups that are present in trazodone, as for example, the amine nitrogens in the piperazine ring. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

The terms “about” or “approximately” as used herein mean within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, a range up to 10%, a range up to 5%, and/or a range up to 1% of a given value. Alternatively, particularly with respect to biological systems or processes, the term can mean within an order of magnitude, e.g., within 5-fold, or within 2-fold, of a value. “About” and “approximately” are used interchangeably herein.

As used herein, the singular forms “a,” “an”, and “the” include plural referents unless the context clearly dictates otherwise.

Unless indicated to the contrary, the singular includes the plural and vice versa.

Further, unless expressly stated to the contrary, “or” refers to an inclusive “or” and not to an exclusive “or.” For example, a condition A or B is satisfied by any one of the following: A is true (or present), and B is false (or not present), A is false (or not present), and B is true (or present), and both A and B are true (or present).

Various ranges of numbers are provided herein. It is to be understood, unless indicated to the contrary, that the range includes the endpoints thereof. For example, if the range is from 25 mg to 50 mg, all the numbers between 25 and 50 mg as well as 25 mg and 50 mg are contemplated to be within the range.

Unless indicated to the contrary, dosages of trazodone or a pharmaceutically salt thereof are provided in mg of active freebase (excluding counterion mass) per about 70 kg human per administration thereof. Accordingly, the amount of trazodone or pharmaceutically acceptable salt thereof can be adjusted accordingly if the human weighs more than or less than about 70 kg.

Trazodone is a triazolopyridinone derivative having the formula:

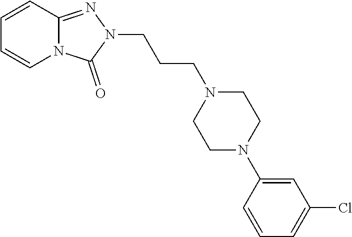 [SMILES: O=c1n(CCCN2CCN(c3cccc(Cl)c3)CC2)nc2ccccn12]

The drug has a complex pharmacology in which it binds to a number of serotonin and adrenergic receptors and exhibits mostly antagonist actions at these targets. Among the targets of trazodone is the 5-HT2A receptor, where the compound binds with nanomolar affinity and acts as an antagonist in vitro. It has been approved by the FDA as an antidepressant for treating major depressive disorders. In addition, it has been marketed as an anxiolytic and sleep aid. It also has been used off-label for treating anxiety, Alzheimer disease, substance abuse, bulimia, and fibromyalgia. It is administered orally and is available in oral tablets of trazodone hydrochloride in strengths of 50 mg, 100 mg, 150 mg, and 300 mg in the US. Typically, oral doses of ≥150 mg/day are used to treat depression, while doses as low as 50 mg/day are used to aid in sleep. Trazodone has not been used by parenteral routes beyond exploratory studies (e.g., intravenous PK studies).

In the context of the present disclosure the term “5-HT2A receptor agonist” is intended to mean any compound or substance that activates the 5-HT2A receptor. The agonist may be a partial or full agonist. Such 5-HT2A receptor agonists include compounds selected from the group consisting of ergolines, tryptamines, phenethylamines, or amphetamines. For example, tryptamines that are 5-HT2A receptor agonists, include, but are not limited to, psilocybin, psilocin, N,N-dimethyltryptamine (DMT), 5-methoxy-N,N-dimethyltryptamine (5-MeO-DMT), N-methyl-N-ethyltryptamine (MET), N-methyl-N-isopropyltryptamine (MIPT), N,N-diethyltryptamine (DET), N,N-diisopropyltryptamine (DIPT), N,N-dipropyltryptamine (DPT), N-ethyl-N-propyltryptamine (EPT), 5-methoxy-N-methyl-N-isopropyltryptamine (5-MeO-MIPT), 5-methoxy-N,N-diisopropyltryptamine (5-MeO-DIPT), 5-methoxy-N-methyl-N-ethyltryptamine (5-MeO-MET), 5-methoxy-N,N-diethyltryptamine (5-MeO-DET), N,N-diallyl-5-methoxytryptamine (5-MeO-DALT), 4-hydroxy-N-methyl-N-ethyltryptamine (4-HO-MET), 4-hydroxy-N-methyl-N-isopropyltryptamine (4-HO-MIPT), 4-hydroxy-N,N-diisopropyltryptamine (4-HO-DIPT), 4-hydroxy-N,N-diethyltryptamine (4-HO-DET), 4-hydroxy-N,N-dipropyltryptamine (4-HO-DPT), 4-hydroxy-N-ethyl-N-propyltryptamine (4-HO-EPT), 4-acetoxy-N-methyl-N-ethyltryptamine (4-AcO-MET), 4-acetoxy-N-methyl-N-isopropyltryptamine (4-AcO-MIPT), 4-acetoxy-N,N-diisopropyltryptamine (4-AcO-DIPT), 4-acetoxy-N,N-diethyltryptamine (4-AcO-DET), 4-acetoxy-N,N-dipropyltryptamine (4-AcO-DPT), 4-acetoxy-N-ethyl-N-propyltryptamine (4-AcO-EPT), 4-acetoxy-N,N-dimethyltryptamine (4-AcO-DMT), alpha-methyltryptamine (AMT), alpha-ethyltryptamine (AET), 5-methoxy-alpha-methyltryptamine (5-MeO-AMT), and the like.

Ergolines that are 5-HT2A receptor agonists include, but are not limited to, lysergic acid amides selected from the group consisting of lysergic acid diethylamide (LSD), lysergic acid 2,4-dimethylazetidide (LSZ), 6-ethyl-6-nor-lysergic acid diethylamide (ETH-LAD), 6-propyl-6-nor-lysergic acid diethylamide (PRO-LAD), 1-acetyl-lysergic acid diethylamide (ALD-52), 1-propionyl-lysergic acid diethylamide (1P-LSD), 1-butyryl-lysergic acid diethylamide (1B-LSD), 1-(cyclopropylmethanoyl)-lysergic acid diethylamide (1 cP-LSD). diethylamide (1B-LSD), and the like.

Phenethylamines that are 5-HT2A receptor agonists include, but are not limited to, mescaline, escaline, proscaline, methallylescaline, allylescaline, 4-bromo-2,5-dimethoxyl)enethylamine (2C-B), 4-chloro-2,5-dimethoxyl)enethylamine (2C-C), 4-iodo-2,5-dimethoxyl)enethylamine (2C-I), 2,5-dimethoxy-4-methylphenethylamine (2C-D), 2-(4-Ethyl-2,5-dimethoxyphenyl)ethanamine (2C-E), 2-(2,5-Dimethoxy-4-propylphenyl)ethan-1-amine (2C-P), 2-[4-(Ethylsulfanyl)-2,5-dimethoxyphenyl]ethan-1-amine (2C-T-2), 2-[2,5-Dimethoxy-4-(propylsulfanyl)phenyl]ethan-1-amine (2C-T-7), 2-(4-iodo-2,5-dimethoxyphenyl)-N-[(2-methoxyphenyl)methyl]ethanamine (25I-NBOMe), 2-(4-bromo-2,5-dimethoxyphenyl)-N-[(2-methoxyphenyl)methyl]ethanamine (25B-NBOMe), 2-(4-chloro-2,5-dimethoxyphenyl)-N-[(2-methoxyphenyl)methyl]ethanamine (25C-NBOMe), 2-(4-methyl-2,5-dimethoxyphenyl)-N-[(2-methoxyphenyl)methyl]ethanamine (25D-NBOMe), 2-(4-ethyl-2,5-dimethoxyphenyl)-N-[(2-methoxyphenyl)methyl]ethanamine (25E-NBOMe), 2-(4-iodo-2,5-dimethoxyphenyl)-N-[(2-hydroxyphenyl)methyl]ethanamine (25I-NBOH), 2-(4-bromo-2,5-dimethoxyphenyl)-N-[(2-hydroxyphenyl)methyl]ethanamine (25B-NBOH), 2-(4-chloro-2,5-dimethoxyphenyl)-N-[(2-hydroxyphenyl)methyl]ethanamine (25C-NBOH), 2-(4-methyl-2,5-dimethoxyphenyl)-N-[(2-hydroxyphenyl)methyl]ethanamine (25D-NBOH), 2-(4-ethyl-2,5-dimethoxyphenyl)-N-[(2-hydroxyphenyl)methyl]ethanamine (25E-NBOH), 2-(4-cyano-2,5-dimethoxyphenyl)-N-[(2-hydroxyphenyl)methyl]ethanamine (25CN-NBOH), and the like.

Amphetamines that are 5-HT2A receptor agonists include, but are not limited to, 2,5-dimethoxy-4-methylamphetamine (DOM), 2,5-dimethoxy-4-bromoamphetamine (DOB), 2,5-dimethoxy-4-chloroamphetamine (DOC,) 2,5-dimethoxy-4-iodoamphetamine (DOI), 2,5-dimethoxy-4-ethylamphetamine (DOET), 2,5-Dimethoxy-4-propylamphetamine (DOPR), and the like.

As used herein, the term “pharmacological effects” refers to the acute behavioral, perceptual, or physiological effects of 5-HT2A agonists occurring while the drug is still present in the bloodstream at a concentration sufficient to induce agonist actions at the 5-HT2A receptor (i.e., a pharmacologically relevant concentration). For example, patients administered 5-HT2A receptor agonists may experience effects including, but not limited to, visual, auditory, or other perceptual disturbances, abnormal thoughts, including suicidal thoughts, anxiety, insomnia, vasoconstriction, elevated blood pressure, or other cardiovascular effects, blurred vision, abnormal heart rate, or nausea. The duration of these effects will necessarily depend on the pharmacokinetic half-life of the particular 5-HT2A agonist in question and the dose administered to the subject. In the context of the present disclosure, the term “pharmacological effects” does not refer to behavioral effects that persist beyond the pharmacokinetic elimination of the drug, for example, antidepressant effects or changes in mood that may last weeks or months after a single administration of a 5-HT2A receptor agonist.

One of the advantages of trazodone and its pharmaceutically acceptable salts is that they are antagonists of both 5-HT2A receptors and alpha-1 adrenergic receptors. Alpha-1 receptor antagonism leads to vasodilation and blood pressure decreases, and acts in concert with the direct reversal effects of the 5-HT2A receptor antagonism of trazodone to reverse the vasoconstrictive and blood pressure elevating effects of 5-HT2A receptor agonists. Without wishing to be bound, it is believed that these two antagonistic properties exhibited by trazodone and its pharmaceutically acceptable salts make trazodone and its pharmaceutically acceptable salts more efficacious than other 5-HT2A receptor antagonists in reversing the vasoconstrictive and blood pressure elevating effects of 5-HT2A receptor agonists, because other more selective 5-HT2A receptor antagonists lack this secondary mechanism.

Trazodone or a pharmaceutically acceptable salt thereof, such as the hydrochloride salt, is administered to patients (animals and humans) in need of such treatment in dosages that will attenuate or reverse the acute pharmacological effects of a 5-HT2A receptor agonist. It will be appreciated that the dose required will vary from patient to patient, taking into consideration various factors, which include, but are not limited to, the route of administration, the dose of the 5-HT2A receptor agonist, the severity of the adverse effects of the 5-HT2A receptor agonist, the nature of the adverse effects of the 5-HT2A receptor agonist, the age, sex, and condition of the patient, concurrent medication or special diets then being followed by the patient, and other factors which those skilled in the art will recognize, with the appropriate dosage ultimately being at the discretion of the attendant physician. For treatment of a patient suffering from adverse effects of a 5-HT2A receptor agonist, trazodone or a pharmaceutically acceptable salt thereof may be administered orally, subcutaneously, intravenously, intramuscularly, by inhalation spray, by vaporization, intranasally, sublingually, buccally, or rectally in dosage unit formulations containing conventional non-toxic pharmaceutically acceptable carriers, adjuvants, and vehicles. Parenteral administration may include subcutaneous, intravenous, or intramuscular injections or infusion techniques. Routes of administration providing rapid absorption and distribution throughout the body and into the central nervous system, for example, intravenous, intramuscular, or intranasal routes, are preferred if rapid reversal of the effects of the 5-HT2A receptor agonist is desired.

Treatment with trazodone commences after administration of the 5-HT2A receptor agonist at any time at which the clinician or subject desires to terminate the pharmacological effects of the 5-HT2A receptor agonist. Treatment may be continued for as long or as short a period as desired. The compositions may be administered on a regimen of, for example, one to four or more times per day. A suitable treatment period may last, for example, for one dose or for a few hours, or for a day or two, but typically not more than 3 days. However, the time frame may vary, on a case-by-case basis, depending on the effectiveness of the treatment, the doses of trazodone administered, and the dose and half-life of the 5-HT2A receptor agonist that is to be reversed. Initially, lower doses may be administered, for example 50 mg or less, and such dosing may be repeated several times per day, or alternatively, a higher dose, such as greater than 50 mg, may be administered to the subject, as described hereinbelow. In an embodiment, the trazodone or pharmaceutically acceptable salt thereof only needs to be administered once. However, if insufficient reversal of the psychedelic effects of the 5-HT2A receptor agonist is achieved or if they recur at a later time after cessation of the administration of trazodone or pharmaceutically acceptable salts thereof, then a second or third dose may be given to increase or reestablish reversal, as needed. A treatment period may terminate when a desired result is achieved, for example a decrease and/or cessation of the adverse effects of the 5-HT2A receptor agonist. In some embodiments, the treatment regimen may be continued for a short time thereafter, such as about 1 to about 3 days to avoid a possible relapse of the symptoms or the adverse effects of the 5-HT2A receptor agonist. A treatment regimen may include a corrective phase, during which a therapeutically effective dose sufficient to provide rapid symptomatic relief is administered, and may be followed by a maintenance phase, during which a lower dose sufficient to prevent a return of symptoms is administered. A suitable maintenance dose is likely to be found in the lower parts of the dose ranges provided herein, but corrective and maintenance doses can readily be established for individual subjects by those of skill in the art without undue experimentation, based on the disclosure herein. Maintenance doses may be employed to maintain remission in subjects, whose symptoms have been previously controlled by other means, including treatments employing other pharmacological agents.

In some embodiments, trazodone or a pharmaceutically acceptable salt thereof is administered to a subject at a dose indicated hereinabove and below in one or more doses. In some embodiments, trazodone or a pharmaceutically acceptable salt thereof is administered to a subject at a dose of about 5 mg/day in one or more doses. In some embodiments, trazodone or a pharmaceutically acceptable salt thereof is administered to a subject at a dose of about 10 mg/day in one or more doses. In some embodiments, trazodone or a pharmaceutically acceptable salt thereof is administered to a subject at a dose of about 15 mg/day in one or more doses. In some embodiments, trazodone or a pharmaceutically acceptable salt thereof is administered to a subject at a dose of about 20 mg/day in one or more doses. In some embodiments, trazodone or a pharmaceutically acceptable salt thereof is administered to a subject at a dose of about 25 mg/day in one or more doses. In some embodiments, trazodone or a pharmaceutically acceptable salt thereof is administered to a subject at a dose of about 30 mg/day in one or more doses. In some embodiments, trazodone or a pharmaceutically acceptable salt thereof is administered to a subject at a dose of about 40 mg/day in one or more doses. In some embodiments, trazodone or a pharmaceutically acceptable salt thereof is administered to a subject at a dose of about 50 mg/day in one or more doses. In some embodiments, trazodone or a pharmaceutically acceptable salt thereof is administered to a subject at a dose of about 75 mg/day in one or more doses. In some embodiments, trazodone or a pharmaceutically acceptable salt thereof is administered to a subject at a dose of about 100 mg/day in one or more doses.

In some embodiments, the subject may be started at a low dose, such as at or below about 10 mg and the dosage is escalated if additional dosage is necessary. In some embodiments, the subject may be started at a high dose, such as at about 100 mg and the dosage is decreased if additional dosage is necessary. In an embodiment, the maximum daily dosage is less than or equal to about 150 mg.

In some embodiments, the dosage of trazodone or a pharmaceutically acceptable salt thereof may range from about 10 mg to about 100 mg per administration per about 70 kg human, for example, if given orally or subcutaneously, and in another embodiment, from about 10 mg to about 50 mg per about 70 kg human. In some embodiments, the dosage is greater than or equal to about 10 mg per about 70 kg human but less than or equal to 100 mg per 70 kg human per administration, while in other embodiments, the dosage may range from about 10 mg to about 50 mg per about 70 kg human per administration. For about a 70 kg human, the dosage of trazodone or pharmaceutically acceptable salt thereof may be, for each administration thereof: 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28, mg, 29 mg, 30 mg, 31 mg, 32 mg, 33 mg, 34 mg, 35 mg, 36 mg, 37 mg, 38 mg, 39 mg, 40 mg, 41 mg, 42 mg, 43, mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, or any value therebetween, being examples.

In some embodiments, dosages of trazodone or a pharmaceutically acceptable salt thereof may range from about 5 mg to about 100 mg per about 70 kg human, for example, when administered intravenously or intramuscularly, for each administration thereof. In some embodiments, the dosage may range for each administration of trazodone or a pharmaceutically acceptable salt thereof from about 10 mg to about 50 mg per about 70 kg human, for each administration, while in another embodiment, it may range from about 5 mg to about 50 mg per about 70 kg human for each administration. For about a 70 kg human, the dosage of trazodone or a pharmaceutically acceptable salt thereof may be, for each administration thereof: 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28, mg, 29 mg, 30 mg, 31 mg, 32 mg, 33 mg, 34 mg, 35 mg, 36 mg, 37 mg, 38 mg, 39 mg, 40 mg, 41 mg, 42 mg, 43, mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, or any value therebetween, being examples.

In some embodiments, dosages of trazodone or a pharmaceutically acceptable salt thereof may range from about 5 mg to about 50 mg per about 70 kg human, for example, when administered intranasally, for each administration thereof. In some embodiments, the dosage may range for each administration of trazodone or a pharmaceutically acceptable salt thereof from about 5 mg to about 25 mg per about 70 kg human, for each administration. For about a 70 kg human, the dosage of trazodone or pharmaceutically acceptable salt thereof may be, for each administration thereof; 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28, mg, 29 mg, 30 mg, 31 mg, 32 mg, 33 mg, 34 mg, 35 mg, 36 mg, 37 mg, 38 mg, 39 mg, 40 mg, 41 mg, 42 mg, 43, mg, 44 mg, 45 mg, 46 mg, 47 mg, 48 mg, 49 mg, 50 mg, or any value therebetween, being examples.

It is to be understood that trazodone may induce drowsiness or sedation, particularly at high doses, for example, doses greater than about 50 mg. Therefore, if the goal of the clinician is to attenuate or reverse the psychedelic effects of a 5-HT2A receptor agonist in a subject without inducing sedation, the minimum possible dose of trazodone necessary to achieve the desired degree of attenuation or reversal should be used. However, it should also be appreciated that in some cases, inducing drowsiness or sedation may be desirable, particularly if the subject is highly agitated or is suffering from insomnia due to the effects of a 5-HT2A receptor agonist.

The dose of trazodone or a pharmaceutically acceptable salt thereof can be determined by measuring the concentration of trazodone in the blood plasma of the patient by techniques known to one of ordinary skill in the art. Using this technique, trazodone or a pharmaceutically acceptable salt thereof is administered to a patient and the plasma concentration is measured after a reasonable amount of time after administration to determine if the plasma concentration of trazodone is within a range that is therapeutically effective and maintained for about 1 hours to about 8 hours. In an embodiment, the plasma concentration is measured within about 5 minutes to about 60 minutes after administration of trazodone or a pharmaceutically acceptable salt thereof. The time point of trazodone measurement in the plasma and the desired duration of time where its plasma concentration is maintained in a therapeutically effective range will depend on the route of administration, dose of trazodone, and the particular 5-HT2A receptor agonist that is to be reversed. For example, intravenous administration will achieve peak plasma concentrations immediately, whereas with oral administration, peak plasma concentrations will not be reached for about 1 hour. Further, higher doses of trazodone will maintain therapeutically effective plasma concentrations for a longer duration of time and therefore, may be more suitable for reversal of 5-HT2A receptor agonists will long half-lives. In an embodiment, the dosage administered to a patient is therapeutically effective if the plasma concentration is in a range from about 100 ng/mL to about 400 ng/mL and in another embodiment, from about 200 ng/mL to about 400 ng/mL. In an embodiment, the plasma concentration of about 100 ng/mL to about 400 ng/mL is achieved at peak effect (Cₘₐₓ). In another embodiment, the plasma concentration of about 100 ng/mL to about 400 ng/mL is maintained for at least about 1 hour, about 2 hours, about 4 hours, about 6 hours, about 8 hours, about 12 hours, or about 24 hours. In an embodiment, the dosage may be adjusted to maintain a plasma concentration ranging from about 200 ng/mL to about 400 ng/mL for at least four hours. For about a 70 kg human, the dosage of trazodone or pharmaceutically acceptable salt thereof may be, for each administration thereof, sufficient to achieve a plasma concentration in ng/mL of about 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, or any value therebetween, being examples.

In some embodiments, trazodone or a pharmaceutically acceptable salt thereof is administered to a patient under the supervision of a healthcare provider.

In some embodiments, trazodone or a pharmaceutically acceptable salt thereof is administered to a patient under the supervision of a healthcare provider at a clinic specializing in the delivery of psychoactive treatments. It may be administered in a low dose, such as a dosage ranging from about 5 mg to 25 mg, or about 5 mg to 50 mg, or at a high dose, such as ranging from about 50 mg to about 100 mg.

In some embodiments, trazodone or a pharmaceutically acceptable salt thereof is administered by a patient on their own at home or otherwise away from the supervision of a healthcare provider.

In some embodiments, trazodone or a pharmaceutically acceptable salt thereof is administered by a patient on his or her own at home or otherwise away from the supervision of a healthcare provider at a low dose, such as a dosage ranging from about 5 mg to 25 mg, or about 5 mg to 50 mg, or at a high dose, such as ranging from about 50 mg to about 100 mg.

It is to be understood that these dosage ranges are exemplary. Dosages below or above the exemplified ranges are also contemplated and within the scope of this disclosure. In addition, the description provided herewith with dose ranges of trazodone or pharmaceutically acceptable salts thereof that should reverse typical doses of 5-HT2A receptor agonists, where typical is defined to mean a dose range that achieves a full psychedelic effect profile, but not an overdose. An overdose of 5-HT2A receptor agonists might require more trazodone or pharmaceutically acceptable salts thereof to reverse the pharmacological effects.

Another aspect of the disclosure provides pharmaceutical compositions comprising compounds as disclosed herein formulated together with a pharmaceutically acceptable carrier. In particular, the present disclosure provides pharmaceutical compositions comprising trazodone or a pharmaceutically acceptable salt thereof formulated together with one or more pharmaceutically acceptable carriers. These formulations include those suitable for oral, rectal, topical, buccal, parenteral (e.g., subcutaneous, intramuscular, intradermal, or intravenous) rectal, vaginal, intranasal, aerosol, or vaporization administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration. Such methods include the step of bringing in association compounds used in the present disclosure or combinations thereof with any auxiliary agent. The auxiliary agent(s), also named accessory ingredient(s), include those conventional in the art, such as carriers, fillers, binders, diluents, disintegrants, lubricants, colorants, flavoring agents, anti-oxidants, and wetting agents. Such auxiliary agents are suitably selected with respect to the intended form and route of administration and as consistent with conventional pharmaceutical practices.

Exemplary pharmaceutical compositions of this disclosure may be used in the form of a pharmaceutical preparation, for example, in solid, semisolid, or liquid form, which contains one or more of the compounds of the disclosure, as an active ingredient, in admixture with an organic or inorganic carrier or excipient suitable for external, enteral, or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease. Pharmaceutical compositions include those suitable for oral, rectal, nasal, topical (including transdermal, buccal, and sublingual), vaginal or parenteral (including subcutaneous, intramuscular, intravenous, and intradermal) administration or administration via an implant. The compositions may be prepared by any method well known in the art of pharmacy.

Pharmaceutical compositions suitable for oral administration may be presented as discrete dosage units such as pills, tablets, dragées or capsules, or as a powder or granules, or as a solution or suspension. The active ingredient may also be presented as a bolus or paste. The compositions can further be processed into a suppository or enema for rectal administration

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of trazodone or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills, and capsules.

In solid dosage forms for oral administration (capsules, tablets, pills, dragees, powders, granules and the like), the subject composition comprising trazodone or a pharmaceutically acceptable salt thereof is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills, and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous, or organic solvents, or mixtures thereof, and powders. Liquid dosage forms for oral administration include pharmaceutically acceptable emulsions, microemulsions, solutions, suspensions, syrups, and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax, or a salicylate, and which is solid at room temperature, but liquid at body temperature and, therefore, will melt in the body cavity and release the active agent.

Dosage forms for transdermal administration of a subject composition include powders, sprays, including nasal sprays, ointments, pastes, creams, lotions, gels, solutions, patches, and inhalants. Trazodone or a pharmaceutically acceptable salt thereof may be mixed under sterile conditions with a pharmaceutically acceptable carrier, and with any preservatives, buffers, or propellants which may be required.

The ointments, pastes, creams, and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions comprising trazodone or a pharmaceutically acceptable salt thereof alternatively be administered by aerosol. This is accomplished by preparing an aqueous aerosol, liposomal preparation or solid particles containing trazodone or a pharmaceutically acceptable salt thereof. A non-aqueous (e.g., fluorocarbon propellant) suspension could be used. Sonic nebulizers may be used because they minimize exposing trazodone or a pharmaceutically acceptable salt thereof to shear, which may result in degradation of trazodone, or a pharmaceutically acceptable salt thereof contained in the subject compositions. Ordinarily, an aqueous aerosol is made by formulating an aqueous solution or suspension of trazodone or pharmaceutically acceptable salt thereof together with conventional pharmaceutically acceptable carriers and stabilizers. The carriers and stabilizers vary, but typically include non-ionic surfactants (Tweens, Pluronics, or polyethylene glycol), innocuous proteins like serum albumin, sorbitan esters, oleic acid, lecithin, amino acids such as glycine, buffers, salts, sugars, or sugar alcohols. Aerosols generally are prepared from isotonic solutions.

Pharmaceutical compositions of this disclosure suitable for parenteral administration comprise trazodone or a pharmaceutically acceptable salt thereof in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants

In another aspect, the disclosure provides enteral pharmaceutical formulations comprising trazodone or a pharmaceutically acceptable salt thereof and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e. g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives of the present disclosure.

A solution comprised of trazodone, or a pharmaceutically acceptable salt thereof may be administered to the patient using an autoinjector commonly used in the art. Auto-injectors are devices which completely or partially replace activities involved in parenteral drug delivery from standard syringes. These activities may include removal of a protective syringe cap, insertion of a needle into a patient's skin, injection of the medicament, removal of the needle, shielding of the needle and preventing reuse of the device. Triggering may be performed by numerous means, for example a trigger button, the action of placing the autoinjector against the skin, or the action of the needle reaching its injection depth.

In some devices the energy to deliver the fluid is provided by a spring. Auto-injectors may be disposable or single use devices which may only be used to deliver one dose of medicament, and which have to be disposed of after use. Other types of auto-injectors may be reusable. Usually, they are arranged to allow a user to load and unload a standard syringe. The reusable auto-injector may be used to perform multiple parenteral drug deliveries, whereas the syringe is disposed after having been spent and unloaded from the auto-injector. The syringe may be packaged with additional parts to provide additional functionality.

The disclosure also provides kits for use by a, e.g., a consumer in need of treatment with trazodone or pharmaceutically acceptable salt thereof. Such kits include a suitable dosage form such as those described above and instructions describing the method of using such dosage form in therapeutically effective amounts, as described hereinabove, to attenuate or reverse the pharmacological effects of a 5-HT2A receptor agonist, for example, hallucinogenic effects. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

An adverse effect of 5-HT2A receptor agonists is insomnia, making it difficult to sleep. Further, another aspect of the disclosure is directed to a method of using trazodone or its pharmaceutically acceptable salts to attenuate the residual wakefulness-promoting effects of 5-HT2A receptor agonists that often persist beyond the time at which the primary perceptual and thought disturbances induced by the agonist have subsided. Users of 5-HT2A receptor agonists frequently report difficulty sleeping that persists beyond the duration of the desired psychedelic experience. Administration of trazodone or pharmaceutically acceptable salts thereof at the doses described above, particularly at higher doses (e.g., about 50 mg to 100 mg), terminates these residual wakefulness-promoting effects in the subject and induces sleep. More specifically, this aspect of the disclosure is directed to a method of promoting or inducing sleep in a subject who is suffering insomnia due to prior administration of a 5-HT2A receptor agonist, comprising administrating to the subject a sleep-inducing, therapeutically effective amount of trazodone or a pharmaceutically acceptable salt thereof at a time when the hallucinogenic effect or other primary pharmacological effect is subsiding. For example, the trazodone is administered at the tail end of the psychedelic experience induced by the 5-HT2A receptor agonist, for example, ~4 h after administration of psilocybin or ~8 h after administration of LSD. The modes of administration, the dosing amounts, the dosing regimens, and the types of pharmaceutical compositions described hereinabove are also applicable, and the discussion is incorporated by reference.

Another aspect of the present disclosure is the utilization of trazodone or a pharmaceutically acceptable salt thereof to reverse hyperthermia induced by serotonin-norepinephrine-dopamine releasing agents (SNDRA), also known as triple releasing agents (TRA). These drugs can be used as recreational drugs to produce euphoriant, entactogenic, and psychostimulant effects. Examples include, but are not limited to, 3,4-methylenedioxymethamphetamine (MDMA), 3,4-methylenedioxyamphetamine (MDA), 4-methylamphetamine, methamphetamine, α-methyltryptamine (αMT), α-ethyltryptamine (α-ET), 5-(2-aminsopropyl)benzofuran (5 APB 6-(2 aminopropyl)benzofuran (6-APB), 1-(benzofuran-5-yl)-N-methylpropan-2-amine (5-MAPB), 1-(benzofuran-6-yl)-N-methylpropan-2-amine (6-MAPB), napthylisopropylamine, 4,4 dimethylaminorex (4,4′-DMAR), 5-iodo-2-aminoindane 5-IAI), 4-fluoroamphetamine (4-FA), 4-fluoromethamphetamine (4-FMA), cathinones, such as mephedrone, methylone, and 3-methmethcathinone (3-MMC), and other TRAs. TRAs, such as MDMA, may induce hyperthermia in persons who ingest them, particularly at high doses, making them dangerous, and sometimes fatal. In addition, it is also believed that the hyperthermia induced by ingestion of these drugs exacerbates the neurotoxic effects of these compounds, based on animal studies. Treatment of a subject suffering from TRA-induced hyperthermia with trazodone or its pharmaceutically acceptable salts reduces body temperature and attenuates secondary neurotoxic effects. The modes of administration, the dosing amounts, the dosing regimens, and the types of pharmaceutical compositions described hereinabove are also applicable, and the discussion is incorporated by reference.

The following examples further illustrate the teachings in the present disclosure.

### Example 1. Trazodone Reversal of Head Twitch Response (HTR)

### Animals

Male C57BL/6 mice (~7-9 weeks old) were used in these studies. Mice were housed 2 per cage under standard temperature- and humidity-controlled laboratory conditions with a 12-hour light/dark cycle. Mice had free access to food and water.

### Drugs and Drug Administration

Trazodone HCl and DOPR HCl were used. Both compounds were dissolved in saline vehicle at a dose volume of 10 mL/kg. Doses were corrected for salt form so that the dose administered represented free base.

### Behavioral Testing

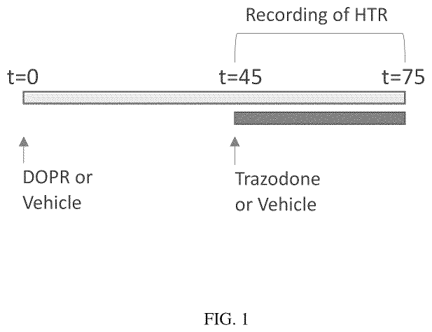

Mice were randomly assigned to 1 of 6 treatment groups consisting of 2 subcutaneous injections (see Table 1), which were administered according to the schedule in FIG. 1. Mice were first dosed with either vehicle (saline) or DOPR (3.2 mg/kg). Forty-five minutes later, mice were dosed with vehicle (saline) or trazodone (1, 1.8, 3.2 or 10 mg/kg) and immediately placed in the testing chamber for 30 min. Behavior was videotaped by a camera (GoPro Hero 9) mounted above the testing chambers. Recorded video was uploaded for analysis of head twitch behavior. Number of head twitch responses (HTRs) was recorded for each animal for each 2-min bin. For each treatment group, the mean number of HTRs in each 2- or 10-min bin, and across the whole 30-min recording period, were analyzed. The ability of trazodone to reduce DOPR-induced HTR was determined by comparing group 2 (DOPR+vehicle) to groups 3-6 (DOPR+trazodone).

TABLE 1
Dosing schedule for HTR experiment.

| | | DOPR Dose | Trazodone Dose | Animals |
|---|---|---|---|---|
| Group | | (mg/kg, SC) | (mg/kg, SC) | per |
| No | Treatment | Time = 0 min | Time = 45 min | group |
| 1 | Vehicle + Vehicle | Normal Saline | Normal Saline | 8 |
| 2 | DOPR + Vehicle | 3.2 | Normal Saline | 8 |
| 3 | DOPR + Trazodone | 3.2 | 1 | 8 |
| 4 | | 3.2 | 1.8 | 8 |
| 5 | | 3.2 | 3.2 | 8 |
| 6 | | 3.2 | 10 | 8 |

### Results

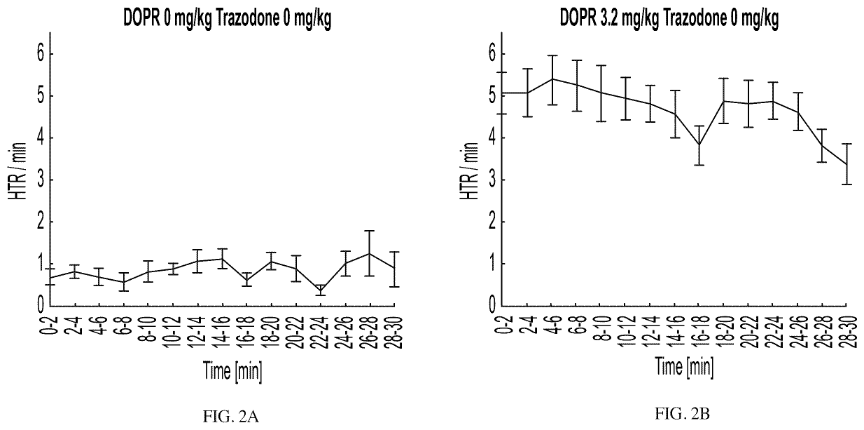

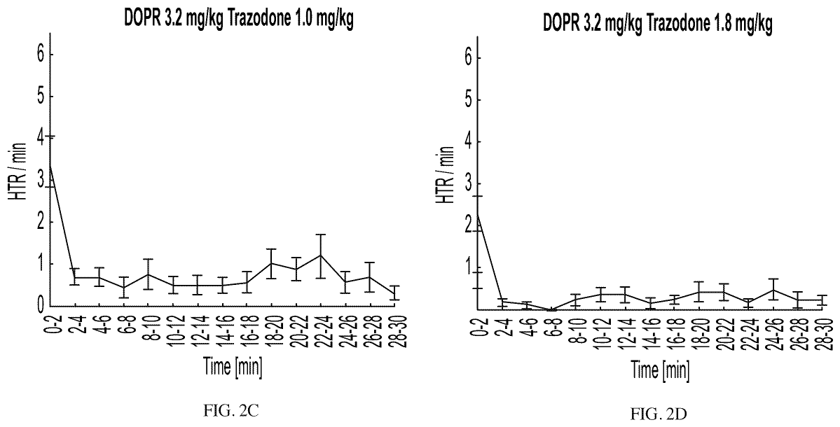

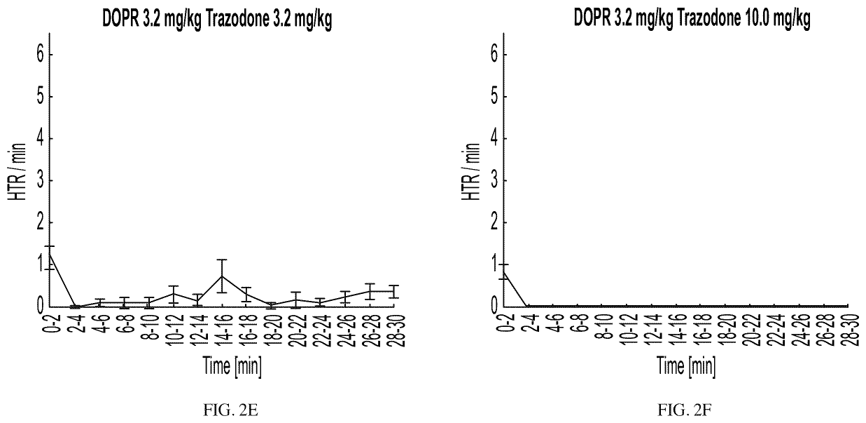

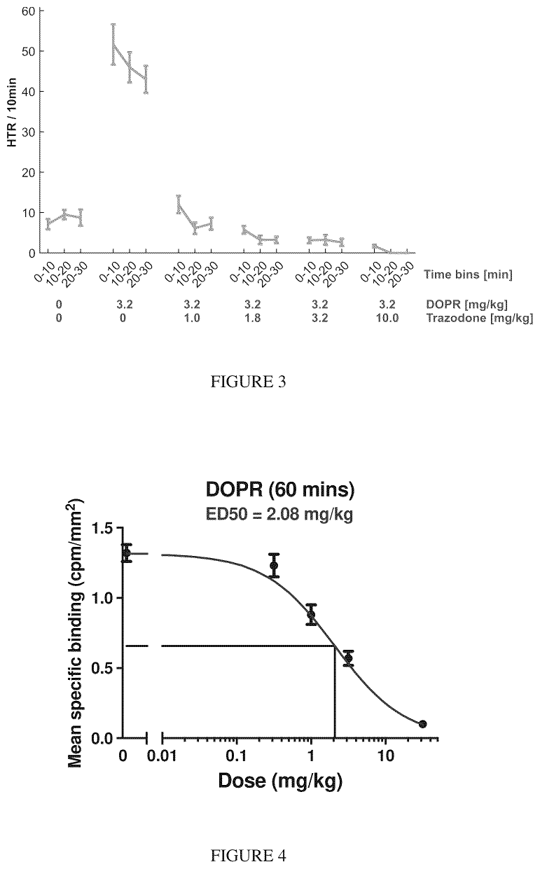

Trazodone, even at the lowest dose tested, fully reversed the HTR induced by DOPR at a dose maximally efficacious in the HTR (see FIGS. 2 and 3).

### Example 2. Occupancy of Cortical 5-HT2A Receptors by DOPR

### Animals

Male C57BL/6 mice, aged 8-12 weeks, were used in these studies. Mice were group housed under standard temperature- and humidity-controlled laboratory conditions with a 12-hour light/dark cycle. Mice had free access to food and water. Animals were randomly assigned to treatment groups.

### Drugs and Drug Administration

DOPR HCl was dissolved in normal saline as the vehicle and administered subcutaneously using a dosing volume of 10 mL/kg body weight. Doses were calculated based on the HCl salt. MDL100,907 [methyl-³H] was purchased from Novandi Chemistry. Ketanserin was purchased from Tocris (UK).

### Sample Collection and Analysis

Mice were dosed with vehicle or DOPR (0.32, 1, 3.2, or 32 mg/kg, SC) and sacrificed at 15 or 60 minutes after dosing (n=5 per dose/time condition) by a rising CO₂ concentration followed by cervical dislocation. Whole brains were removed, rinsed briefly with saline, and blot dried. A coronal cut was made at the level of the optic chiasm to produce an anterior brain block containing the frontal cortex (according to the Mouse Brain in Stereotaxic Coordinates, Paxinos and Franklin, second edition). The anterior brain block was then cut down the midline, both blocks were placed onto cork disks, covered with OCT embedding matrix (KMA-0100-00A, Cell Path), rapidly frozen in isopentane (cooled to −20/−30° C.) and stored at −20° C. for sectioning and ex vivo autoradiography. Coronal sections containing the frontal cortex were be cut (20 μm thick) using a cryostat and thaw mounted onto Superfrost plus slides and the slides were stored at −20° C. until the day of assay.

Slides containing brain sections were warmed to room temperature and sections were isolated using a DAKO pen and incubated at room temperature for 30 minutes in 200 μL of assay buffer containing 0.2 nM [³H]MDL100,907 (total binding) or 0.2 nM [³H]MDL100,907 and 10 M ketanserin (non-specific binding). Solutions were removed from the sections by aspiration and the slides were washed for two consecutive 5-minute periods in ice-cold wash buffer. The slides were then rinsed briefly in ice-cold distilled water to remove buffer salts and allowed to air dry. The assay and wash buffer consisted of 50 mM Tris, pH 7.4. The binding assay was performed on two separate occasions.

The waxy residue of the DAKO pen was removed with xylene to prevent sparks in the β-imager. Lint-free tissue and an air duster was used to remove any dust particles from the microscope slides. To make the slides conductive, copper foil tape was adhered to the free side of the microscope slides. The slides were placed in the β-imager and the levels of bound radioactivity in the sections were directly determined by counting the number of β-particles emerging from the delineated area using the M3 vision program (BioSpace). Data was collected from the brain sections over 16 hours and expressed in counts per minute per square millimetre (cpm/mm²). A value for specific binding (cpm/mm²) was generated by the subtraction of mean non-specific binding (cpm/mm²) from mean total binding (cpm/mm²) for each animal.

### Results

The 5-HT2A receptor occupancy achieved by DOPR in mice was determined using ex vivo receptor occupancy with the selective 5-HT2A receptor antagonist tracer [³H]MDL100,907. Administration of DOPR resulted in dose-dependent reductions in the amount of [³H]MDL100,907 binding, indicating increasing 5-HT2A receptor occupancy by DOPR (FIG. 4). The dose of DOPR calculated to occupy 50% of 5-HT2A receptors was 2.08 mg/kg. The dose of DOPR used in the HTR experiments of Example 1 (3.2 mg/kg, SC) achieved 57% occupancy of 5-HT2A receptors at the approximate time point of HTR measurement. The 5-HT2A receptor occupancies determined at other doses and time points are presented in Table 2.

TABLE 2
Occupancy of 5-HT2A receptors by DOPR.

| | | | Specific | | |
|---|---|---|---|---|---|
| DOPR Dose | | Mean | Binding (% | % | P |
| (mg/kg, SC) | Time | (SEM) | of Vehicle) | Occupancy | value |
| Vehicle | 15 min | 1.32 (0.06) | 100 | 0 | — |
| 0.32 | | 1.39 (0.06) | 105 | −5 | 1 |
| 1 | | 1.38 (0.06) | 104 | −4 | 1 |
| 3.2 | | 0.93 (0.09) | 71 | 29 | <0.001 |
| 32 | | 0.23 (0.04) | 18 | 82 | <0.001 |
| 0.32 | 60 min | 1.23 (0.08) | 93 | 7 | 0.421 |
| 1 | | 0.88 (0.07) | 67 | 33 | <0.001 |
| 3.2 | | 0.57 (0.05) | 43 | 57 | <0.001 |
| 32 | | 0.10 (0.01) | 8 | 92 | <0.001 |

### Example 3. Measurement of Plasma and Brain Concentrations of Trazodone in Mice Animals

Male C57BL/6 mice, aged 8-12 weeks, were used in these studies. Mice were housed 4 per cage under standard temperature- and humidity-controlled laboratory conditions with a 12-hour light/dark cycle. Mice had free access to food and water but were fasted from 4 h prior to and until 2 h after dosing. Animals were randomly assigned to treatment groups.

### Drugs and Drug Administration

Trazodone HCl was prepared using normal saline as the vehicle and administered subcutaneously using a dosing volume of 10 mL/kg body weight. Doses were corrected for salt form so that the dose administered represented free base.

### Sample Collection and Bioanalysis

Twelve mice were dosed subcutaneously with each dose level of trazodone (1, 3.2, or 10 mg/kg). Blood samples (approximately 60 μL) were collected under light isoflurane anesthesia (Surgivet®) from the retro orbital plexus at 5, 15, or 25 min (4 animals per time point). Immediately after blood collection, plasma was harvested by centrifugation at 4,000 rpm for 10 min at 4° C. and samples were stored at −70±10° C. until bioanalysis. Following blood collection, animals were immediately sacrificed, the abdominal vena-cava was cut open, the whole body was perfused from the heart using 10 mL of normal saline, and brain samples were collected from all animals. After isolation, brain samples were rinsed three times in ice-cold normal saline (for 5-10 seconds/rinse using ~5-10 mL normal saline in a disposable petri dish for each rinse) and dried on blotting paper. Brain samples were homogenized using ice-cold phosphate-buffered saline (pH 7.4). Total homogenate volume was three times the tissue weight. All homogenates were stored at −70±10° C. until bioanalysis. For bioanalysis, 20 μL aliquots of plasma/brain study samples or spiked plasma/brain calibration standards were added to individual pre-labeled micro-centrifuge tubes followed by 200 μL of an internal standard solution (cetirizine, 50 ng/mL in acetonitrile) except for blanks, where 200 μL of acetonitrile was added. Samples were vortexed for 5 minutes and then centrifuged for 10 minutes at 4,000 rpm at 4° C. Following centrifugation, 200 μL of each clear supernatant was transferred to a 96-well plate and analyzed with a fit-for-purpose LC-MS/MS method, with authentic samples of each analyte used for calibration and identification.

### Results

Concentrations of trazodone were measured in the plasma and brains of mice after subcutaneous (SC) dosing at time points corresponding to those analyzed in the HTR reversal experiment (Example 1). The results are presented in Table 3.

TABLE 3
Plasma and brain concentrations of trazodone in
mice (n = 4 per dose, per time point) at
the indicated time points after the indicated dose.

| Dose | | | | | | |
|---|---|---|---|---|---|---|
| Trazodone | Plasma concentration (ng/mL) | | | Brain Concentration (ng/g) | | |
| (mg/kg, SC) | 5 min | 15 min | 25 min | 5 min | 15 min | 25 min |
| 1 | 800 | 504 | 369 | 700 | 584 | 255 |
| 3.2 | 1479 | 1347 | 1021 | 1747 | 1600 | 674 |
| 10 | 3491 | 2870 | 1909 | 7930 | 5097 | 3579 |

### Example 4. Measurement of Plasma Protein Binding of Trazodone in Mouse and Human Plasma

### Method

A 1 mM stock solution of trazadone was prepared in DMSO and diluted 200-fold in mouse or human plasma to yield a final concentration of 5 μM. The final DMSO concentration was 0.5%. Rapid equilibrium dialysis (RED) was performed with a RED device containing a dialysis membrane with a molecular weight cut-off of 8,000 Daltons. Each dialysis insert contains two chambers. The red chamber is for plasma while the white chamber is for buffer.

For each dialysis insert, aliquots (200 μL) of positive control or test compound at 5 μM (n=2) were separately added to the plasma chamber and 350 μL of phosphate buffered saline (pH 7.4) was added to the buffer chamber. After sealing the RED device with an adhesive film, dialysis was performed in an incubator at 37° C. with shaking at 300 RPM for 4 hours. To assess recovery and stability, 50 μL aliquots of positive control and test compound were added to 96-deep well plates and quenched with 400 μL acetonitrile (for 0 min), while comparator aliquots were incubated at 37° C. for 4 hours along with the RED device.

Following dialysis, an aliquot of 50 μL was removed from each well (both plasma and buffer side) and diluted with an equal volume of the opposite matrix (dialyzed with the other matrix) to nullify the matrix effect. Similarly, 50 μL of buffer was added to recovery and stability samples. An aliquot of 100 μL was submitted for LC-MS/MS analysis.

Aliquots (50 μL) of test samples were protein precipitated with 400 μL of acetonitrile containing internal standard (glipizide) and vortexed for 5 minutes. The samples were centrifuged at 4,000 RPM at 4° C. for 10 min and 100 μL aliquots of supernatant were submitted for LC-MS/MS analysis of parent compound using a fit-for-purpose LC-MS/MS method.

### Results

The bound and unbound fractions of trazodone in mouse and human plasma were determined using equilibrium dialysis. Trazodone was 94.4% and 90.2% bound (5.6% and 9.8% unbound) in mouse and human plasma, respectively, after 4 hours of incubation.

### Explanation of Examples 1-4

The experiments described in EXAMPLES 1 and 3 were conducted to demonstrate the ability of trazodone to reverse the head-twitch response (HTR) in mice (a rodent correlate of hallucinogenic effects in man) and establish the dose levels and exposures necessary to induce such reversal effects. Further, additional studies described in EXAMPLES 2 and 4 were conducted to allow extrapolation of the effective dose and exposure levels determined in rodents to humans.

First, the prototypical 5-HT2A receptor agonist 2,5-dimethoxy-4-propylamphetamine (DOPR) was used to induce HTR in mice and the ability of several doses of trazodone administered after DOPR to reverse that HTR was determined (see Example 1). The dose (3.2 mg/kg, SC) of DOPR selected for these experiments is maximally efficacious for induction of HTR by this compound (higher and lower doses result in fewer HTRs) and results in 5-HT2A receptor occupancy of 57% at 1 h post dosing (the pharmacokinetic Tₘₐₓ after SC administration). In addition to inducing maximal hallucinogenic-like effects in mice, the level of occupancy achieved by this dose of DOPR in mice (see Example 2) is similar to the 60-70% reported to be necessary to induce intense psychedelic experiences after psilocybin administration in humans. Accordingly, an exposure level (and dose allometrically scaled for species) of trazodone able to fully reverse the effects of DOPR at the selected dose is also able to fully reverse the effects of any 5-HT2A receptor agonist at a fully psychedelic dose in both mice and humans.

Mice were first administered DOPR (3.2 mg/kg, SC). After 45 minutes, mice were administered (SC) one of a range of doses of trazodone or vehicle and HTRs were counted via video recording for the 30-minute period following trazodone dosing (45-75 minutes after DOPR administration; FIG. 1). The time point for trazodone administration and HTR recording was selected so that the recording window would center on the time of maximal 5-HT2A receptor occupancy and HTR for DOPR (60 minutes post administration) and also such that the speed of onset of trazodone's reversal effect could be determined. All doses of trazodone tested, including the lowest dose, were found to rapidly (within 2 minutes) and fully reverse the HTR of DOPR and the reversal effect persisted for the entire duration of the 30-minute recording period (FIG. 2). Further, higher doses of trazodone suppressed HTR below the baseline level observed in vehicle pre-treated animals.

Effects were also quantified in 10-minute bins (0-10, 10-20, and 20-30 minutes post trazodone administration), which showed the same rapid and robust HTR reversal effect, even at the lowest dose (FIG. 3).

In separate PK experiments, plasma, and brain concentrations of trazodone at 5, 15, and 25 minutes post SC dosing were determined, coinciding with the midpoints of the three 10-minute bins analyzed in FIG. 3 (see Example 3).

Since even the lowest dose of trazodone was sufficient to maintain full reversal of the DOPR-induced HTR in the 20-30-minute time bin (FIG. 3), where trazodone concentrations were lowest (see Example 3), it is concluded that the minimum efficacious concentration (MEC) in plasma for reversal of the hallucinogenic effects of a fully psychedelic dose of a 5-HT2A receptor agonist by trazodone is ≤~369 ng/mL (the plasma concentration 25 minutes after 1 mg/kg, SC trazodone).

Using the above plasma MEC in mice and having knowledge of the plasma pharmacokinetics of trazodone in humans, one can estimate the dose of trazodone required to reverse the hallucinogenic effects of a 5-HT2A receptor agonist at a typical, fully psychedelic, but not excessive, dose (e.g., −25 mg of psilocybin orally) in humans. According to the free drug hypothesis, the pharmacological effects of a compound in vivo are mediated by the free, unbound concentration of that compound. Therefore, it is important to account for interspecies differences in plasma protein binding when predicting human dosing on the basis of the above mouse findings. Using equilibrium dialysis, the unbound fraction of trazodone in mouse plasma was found to be 5.6%, while in human plasma it was found to be 9.8% (see Example 4). Accordingly, the total plasma MEC of −369 ng/mL determined in mice corresponds to a free plasma concentration of −21 ng/mL (~369 mg/mL*0.056). This in turn corresponds to a total human plasma MEC of −214 ng/mL (~21 ng/mL/0.098). Based on the data, human doses of trazodone achieving total plasma concentrations ≥~214 ng/mL are therefore expected to reverse the hallucinogenic effects of any 5-HT2A receptor agonist at a typical, fully psychedelic dose as long as the plasma concentration of trazodone remains above this MEC. The concept of a “typical, fully psychedelic dose” should be understood to mean the minimum dose required to induce a psychedelic experience in a typical human subject that is subjectively reported to be intense and evoke the typical cognitive phenomena characteristic of the psychedelic state (e.g., visual, auditory, or other perceptual disturbances and substantial changes in thought patterns).

Examples of fully psychedelic doses of common 5-HT2A receptor agonists, which would be reversible at the MEC of ~214 ng/mL, are listed in Table 4. Typical, fully psychedelic dose ranges of other 5-HT2A receptor agonists can be found in PiHKAL: A Chemical Love Story (Shulgin, 1991), the contents of which are incorporated herein by reference.

TABLE 4
Typical fully psychedelic doses of common
5-HT2A receptor agonists.

| | Typical Fully Psychedelic Dose Range |
|---|---|
| Compound | (~mg per ~70 kg human, route) |
| psilocybin | 15-25, oral |
| psilocin | 15-25, oral |
| DMT | 20-40, smoked |
| 5-MeO-DMT | 5-10, smoked |
| LSD | 0.1-0.2, oral |
| mescaline | 200-400, oral |
| 2C-B | 15-25, oral |
| 2C-E | 10-20, oral |
| DOM | 3-5, oral |

Using the human pharmacokinetic parameters of trazodone (Table 5), the minimum efficacious dose (MED) required to achieve this MEC for reversal of 5-HT2A-induced subjective effects, including visual, auditory, and thought disturbances, by different routes of administration can be estimated.

TABLE 5
Human plasma pharmacokinetic parameters of trazodone collected
from the literature. Cₘₐₓ values are reported as ng/ml in plasma
per mg of trazodone dosed, assuming linear pharmacokinetics.

| Dose | | Cₘₐₓ (ng/mL | Tₘₐₓ | T_{1/2} | |
|---|---|---|---|---|---|
| (mg) | Route | per mg dosed) | (h) | (h) | Reference |
| 25 | IV | NR | NA | 5.3 | Greenblatt, 1987 |
| 90 | IV | NR | NA | 7.3 | Nilsen, 1992 |
| | Mean: | | | 6.3 | |
| 50 | PC | 18.8 | 1.02 | 5.2 | Greenblatt, 1987 |
| 50 | PO | 17.16 | 0.9 | 13.3 | Gammans, 1984 |
| 100 | PO | 18.8 | 1.34 | 7.8 | Nilsen, 1992 |
| 100 | PO | 16 | 2 | 6.0 | Bayer, 1983 |
| 100 | PO | 15.47 | 2.25 | 13.1 | Kale, 2015 |
| | Mean: | 17.2 | 1.5 | 9.1 | |

NA = not applicable;
NR = not reported

By the oral route, the MED is estimated to be −12 mg (~214 ng/mL/~17.2 ng/mL/mg). By the intravenous route, the MED is estimated to be <12 mg given the higher peak concentrations achieved following intravenous bolus administration compared to oral administration. Peak reversal effect after oral trazodone administration is estimated to occur at ~1-2 h, corresponding to the Tₘₐₓ of trazodone. Likewise, peak reversal effect after trazodone administration as an intravenous bolus occurs almost instantaneously (<5 minutes). Administration of trazodone by other routes, such as intranasal, intramuscular, or subcutaneous is also contemplated and will achieve the same effect. Sublingual and buccal routes can also be used but may not be as desirable due to slower onset of action. In the case of these other routes, it is to be understood that the MED and rate of onset will depend on A) the PK of trazodone after administration by such routes and B) the specific formulation used, and therefore, the dose will need to be selected to achieve a total plasma concentration of ≥~214 ng/mL. Rate of onset by intranasal and intramuscular routes should be faster than oral administration, reaching peak reversal effect in ~15-20 minutes. The dose required to achieve a given peak magnitude of reversal (plasma Cₘₐₓ) by these routes should be intermediate between oral and intravenous routes.

Assuming that the desired degree of hallucinogenic reversal (e.g., complete reversal) is achieved after the initial administration of trazodone, administration of additional doses of trazodone should not be needed to maintain the effect level assuming that A) the trazodone is dosed after Tₘₐₓ of the 5-HT2A receptor agonist and B) the half-life of the 5-HT2A receptor agonist being reversed is less than that of trazodone (~6-12 h). It should be understood that in cases where trazodone is dosed before Tₘₐₓ of the 5-HT2A receptor agonist, such that plasma concentrations of the agonist are still increasing, recurrence of hallucinogenic effects may be possible if plasma concentrations of the agonist rise above those that can be overcome by the dose of trazodone administered. Likewise, for 5-HT2A receptor agonists with exceptionally long half-lives (>T_{1/2} of trazodone), re-administration of trazodone may be necessary to maintain reversal as trazodone would be eliminated faster than the competing agonist. Furthermore, given the competitive nature of trazodone's 5-HT2A receptor antagonism, it is to be understood that reversal of an exceptionally high dose of a given 5-HT2A receptor agonist (e.g., doses substantially higher than those described in Table 4) will necessarily require higher doses of trazodone that achieve plasma concentrations greater than the MEC outlined above. However, even low, or normal doses of trazodone may provide partial reversal of the psychedelic effect in such cases, which is likely to be beneficial to the subject in terms of reducing anxiety and physiological risks even in the absence of complete reversal. Likewise, reversal of a lower dose of a 5-HT2A receptor agonist may require a lower dose of trazodone. Lastly, the trazodone may be administered at any time after administration of the 5-HT2A receptor agonist at the discretion of the subject or the healthcare provider, whether to abort an unpleasant psychological experience as reported by the subject, to attenuate dangerous cardiovascular side effects, or simply to shorten the duration of a psychedelic therapy session for convenience.

The above preferred embodiments and examples were given to illustrate the scope and spirit of the present invention. These embodiments and examples will make apparent to those skilled in the art other embodiments and examples. The other embodiments and examples are within the contemplation of the present invention. Therefore, the present invention should be limited only by the amended claims.

1. A method of reversing or abating the pharmacological effects of a 5-HT2A receptor agonist in a subject comprising administering to said subject in need of such treatment a therapeutically effective amount of trazodone or a pharmaceutically acceptable salt thereof.

2. The method of claim 1, wherein the effects reversed comprise visual, auditory, or other perceptual disturbances.

3. The method of claim 1, wherein the effects reversed comprise abnormal thoughts.

4. The method of claim 1, wherein the effects reversed comprise vasoconstriction, blood pressure increases, or other cardiovascular effects.

5. The method of claim 1, wherein the effects reversed comprise nausea.

6. The method of claim 1, wherein the effects reversed comprise insomnia.

7. The method of claim 1 wherein the pharmacological effect is hallucination.

8. A method of promoting or inducing sleep in a subject who continues to suffer insomnia following administration of a 5-HT2A receptor agonist after the primary perceptual effects of said agonist have subsided, said method comprising administrating to the subject a therapeutically effective amount of trazodone or a pharmaceutically acceptable salt thereof.

9. The method of claim 1, wherein the 5-HT2A receptor agonist to be reversed is selected from the group consisting of an ergoline, a tryptamine, a phenethylamine, or an amphetamine.

10. The method of claim 9, wherein the tryptamine is selected from the group consisting of psilocybin, psilocin, N,N-dimethyltryptamine (DMT), 5-methoxy-N,N-dimethyltryptamine (5-MeO-DMT), N-methyl-N-ethyltryptamine (MET), N-methyl-N-isopropyltryptamine (MIPT), N,N-diethyltryptamine (DET), N,N-diisopropyltryptamine (DIPT), N,N-dipropyltryptamine (DPT), N-ethyl-N-propyltryptamine (EPT), 5-methoxy-N-methyl-N-isopropyltryptamine (5-MeO-MIPT), 5-methoxy-N,N-diisopropyltryptamine (5-MeO-DIPT), 5-methoxy-N-methyl-N-ethyltryptamine (5-MeO-MET), 5-methoxy-N,N-diethyltryptamine (5-MeO-DET), N,N-diallyl-5-methoxytryptamine (5-MeO-DALT), 4-hydroxy-N-methyl-N-ethyltryptamine (4-HO-MET), 4-hydroxy-N-methyl-N-isopropyltryptamine (4-HO-MIPT), 4-hydroxy-N,N-diisopropyltryptamine (4-HO-DIPT), 4-hydroxy-N,N-diethyltryptamine (4-HO-DET), 4-hydroxy-N,N-dipropyltryptamine (4-HO-DPT), 4-hydroxy-N-ethyl-N-propyltryptamine (4-HO-EPT), 4-acetoxy-N-methyl-N-ethyltryptamine (4-AcO-MET), 4-acetoxy-N-methyl-N-isopropyltryptamine (4-AcO-MIPT), 4-acetoxy-N,N-diisopropyltryptamine (4-AcO-DIPT), 4-acetoxy-N,N-diethyltryptamine (4-AcO-DET), 4-acetoxy-N,N-dipropyltryptamine (4-AcO-DPT), 4-acetoxy-N-ethyl-N-propyltryptamine (4-AcO-EPT), 4-acetoxy-N,N-dimethyltryptamine (4-AcO-DMT), alpha-methyltryptamine (AMT), alpha-ethyltryptamine (AET), and 5-methoxy-alpha-methyltryptamine (5-MeO-AMT).

11. The method of claim 9, wherein the ergoline is a lysergic acid amide selected from the group consisting of lysergic acid diethylamide (LSD), lysergic acid 2,4-dimethylazetidide (LSZ), 6-ethyl-6-nor-lysergic acid diethylamide (ETH-LAD), 6-propyl-6-nor-lysergic acid diethylamide (PRO-LAD), 6-allyl-6-nor-lysergic acid diethylamide (AL-LAD), 1-acetyl-lysergic acid diethylamide (ALD-52), 1-propionyl-lysergic acid diethylamide (1P-LSD), 1-butyryl-lysergic acid diethylamide (1B-LSD), and 1-(cyclopropylmethanoyl)-lysergic acid diethylamide (1 cP-LSD).

12. The method of claim 9, wherein the phenethylamine is selected from the group consisting of mescaline, escaline, proscaline, methallylescaline, allylescaline, 4-bromo-2,5-dimethoxyl)enethylamine (2C-B), 4-chloro-2,5-dimethoxyl)enethylamine (2C-C), 4-iodo-2,5-dimethoxyl)enethylamine (2C-I), 2,5-dimethoxy-4-methylphenethylamine (2C-D), 2-(4-Ethyl-2,5-dimethoxyphenyl)ethanamine (2C-E), 2-(2,5-Dimethoxy-4-propylphenyl)ethan-1-amine (2C-P), 2-[4-(Ethylsulfanyl)-2,5-dimethoxyphenyl]ethan-1-amine (2C-T-2), 2-[2,5-Dimethoxy-4-(propylsulfanyl)phenyl]ethan-1-amine (2C-T-7), 2-(4-iodo-2,5-dimethoxyphenyl)-N-[(2-methoxyphenyl)methyl]ethanamine (25I-NBOMe), 2-(4-bromo-2,5-dimethoxyphenyl)-N-[(2-methoxyphenyl)methyl]ethanamine (25B-NBOMe), 2-(4-chloro-2,5-dimethoxyphenyl)-N-[(2-methoxyphenyl)methyl]ethanamine (25C-NBOMe), 2-(4-methyl-2,5-dimethoxyphenyl)-N-[(2-methoxyphenyl)methyl]ethanamine (25D-NBOMe), 2-(4-ethyl-2,5-dimethoxyphenyl)-N-[(2-methoxyphenyl)methyl]ethanamine (25E-NBOMe), 2-(4-iodo-2,5-dimethoxyphenyl)-N-[(2-hydroxyphenyl)methyl]ethanamine (25I-NBOH), 2-(4-bromo-2,5-dimethoxyphenyl)-N-[(2-hydroxyphenyl)methyl]ethanamine (25B-NBOH), 2-(4-chloro-2,5-dimethoxyphenyl)-N-[(2-hydroxyphenyl)methyl]ethanamine (25C-NBOH), 2-(4-methyl-2,5-dimethoxyphenyl)-N-[(2-hydroxyphenyl)methyl]ethanamine (25D-NBOH), 2-(4-ethyl-2,5-dimethoxyphenyl)-N-[(2-hydroxyphenyl)methyl]ethanamine (25E-NBOH), and 2-(4-cyano-2,5-dimethoxyphenyl)-N-[(2-hydroxyphenyl)methyl]ethanamine (25CN-NBOH).

13. The method of claim 9, wherein the amphetamine is selected from the group consisting of 2,5-dimethoxy-4-methylamphetamine (DOM), 2,5-dimethoxy-4-bromoamphetamine (DOB), 2,5-dimethoxy-4-chloroamphetamine (DOC,) 2,5-dimethoxy-4-iodoamphetamine (DOI), 2,5-dimethoxy-4-ethylamphetamine (DOET), and 2,5-Dimethoxy-4-propylamphetamine (DOPR).

14. The method of claim 9, wherein the 5-HT2A receptor agonist is selected from the group consisting of psilocybin, 4-AcO-DMT, psilocin, DMT, 5-MeO-DMT, LSD, mescaline, 2C-B, 2C-E, 2C-T-2, 2C-T-7, and DOM.

15. A method of treating hyperthermia induced by a serotonin-norepinephrine-dopamine releasing agent comprising administering a therapeutically effective amount of trazodone or a pharmaceutically acceptable salt thereof to a subject in need of treatment.

16. The method of claim 15, wherein the serotonin-norepinephrine-dopamine releasing agent is 3,4-methylenedioxymethamphetamine (MDMA), 3,4-methylenedioxyamphetamine (MDA), 4-methylamphetamine, methamphetamine, α-methyltryptamine (αMT), α-ethyltryptamine (α-ET), 5-(2-aminopropyl)benzofuran (5-APB), 6-(2-aminopropyl)benzofuran (6-APB), 1-(benzofuran-5-yl)-N-methylpropan-2-amine (5-MAPB), 1-(benzofuran-6-yl)-N-methylpropan-2-amine (6-MAPB), naphthylisopropylamine, 4,4′-dimethylaminorex (4,4′-DMAR), 5-iodo-2-aminoindane (5-IAI), 4-fluoroamphetamine (4-FA), 4-fluoromethamphetamine (4-FMA), mephedrone, methylone, or 3-methylmethcathinone (3-MMC).

17. The method according to claim 15, wherein the serotonin-norepinephrine-dopamine releasing agent is 3,4-methylenedioxymethamphetamine.

18. The method of claim 1, wherein trazodone or a pharmaceutically acceptable salt thereof is administered orally.

19. The method of claim 18, wherein the dose of trazodone or a pharmaceutically acceptable salt thereof ranges from about 10 mg to about 100 mg per about 70 kg human.

20. The method of claim 18, wherein the dose of trazodone or a pharmaceutically acceptable salt thereof ranges from about 10 mg to about 50 mg per about 70 kg human.

21. The method of claim 1, wherein trazodone or a pharmaceutically acceptable salt thereof is administered intravenously.

22. The method of claim 21, wherein the dose of trazodone or a pharmaceutically acceptable salt thereof ranges from about 5 mg to about 100 mg per about 70 kg human.

23. The method of claim 21, wherein the dose of trazodone or a pharmaceutically acceptable salt thereof ranges from about 5 mg to about 50 mg per about 70 kg human.

24. The method of claim 1, wherein trazodone or a pharmaceutically acceptable salt thereof is administered intranasally.

25. The method of claim 24, wherein the dose of trazodone or a pharmaceutically acceptable salt thereof ranges from about 5 mg to about 50 mg per about 70 kg human.

26. The method of claim 24, wherein the dose of trazodone or a pharmaceutically acceptable salt thereof ranges from about 5 mg to about 25 mg per about 70 kg human.

27. The method of claim 1, wherein trazodone is administered intramuscularly.

28. The method of claim 27, wherein the dose of trazodone or a pharmaceutically acceptable salt thereof ranges from about 5 mg to about 100 mg per about 70 kg human.

29. The method of claim 27, wherein the dose of trazodone or a pharmaceutically acceptable salt thereof ranges from about 5 mg to about 50 mg per about 70 kg human.

30. The method of claim 1, wherein trazodone or a pharmaceutically acceptable salt thereof is administered subcutaneously.

31. The method of claim 30, wherein the dose of trazodone or a pharmaceutically acceptable salt thereof ranges from about 10 mg to about 100 mg per about 70 kg human.

32. The method of claim 30, wherein the dose of trazodone or a pharmaceutically acceptable salt thereof ranges from about 10 mg to about 50 mg per about 70 kg human.

33. The method of claim 1, wherein the dose of trazodone or a pharmaceutically acceptable salt thereof is sufficient to achieve a peak plasma concentration of at least 100 ng/mL.

34. The method of claim 1, wherein the dose of trazodone or a pharmaceutically acceptable salt thereof is sufficient to achieve a peak plasma concentration of at least 200 ng/mL.

35. The method of claim 1, wherein the dose of trazodone or a pharmaceutically acceptable salt thereof is sufficient to achieve a peak plasma concentration of about 100 ng/mL to about 400 ng/mL.

36. The method of claim 1, wherein the dose of trazodone or a pharmaceutically acceptable salt thereof is sufficient to maintain a plasma concentration of at least 200 ng/mL for at least 4 hours.

37. The method of claim 1, wherein trazodone or a pharmaceutically acceptable salt thereof is administered more than once per day to maintain reversal of a long-acting 5-HT2A receptor agonist.

38. The method of claim 1, wherein the pharmaceutically acceptable salt of trazodone is trazodone hydrochloride.
